(19) United States
(12) Patent Application Publication
(10) Pub. No.: US 2009/0041829 A1
(43) Pub. Date: Feb. 12, 2009

(54) PHARMACEUTICAL DOSAGE FORMS COMPRISING A LIPID PHASE

(75) Inventors: Bengt Herslof, Stockholm (SE); Henri Hansson, Helsingborg (SE); Per Tingvall, Norberg (SE)

Correspondence Address: DICKSTEIN SHAPIRO LLP, 1177 AVENUE OF THE AMERICAS (6TH AVENUE), NEW YORK, NY, 10036-2714, US

(73) Assignee: Galenica Technology AB, Malmo (SE)

(21) Appl. No.: 11/912,783

(22) PCT Filed: Apr. 27, 2006
(86) PCT No.: PCT/SE2006/050102

§ 371 Date: Jan. 23, 2008

(30) Foreign Application Priority Data
Apr. 28, 2005 (SE) 0500966-7

(51) Int. Cl. A61K 9/02; A61K 9/16; A61K 9/52

(52) U.S. Cl. 424/436; 424/465; 424/452

(57) ABSTRACT

A tablet for oral administration comprises a lipid phase, comprised to 80% by weight or more by a mixture of (a) triglyceride, (b) mono- or/and diglyceride, and (c) cell membrane lipid; (d) one or more pharmacologically active agents dissolved or dispersed in the lipid phase; (e) water and/or ethanol; and (f) an absorption controlling amount of particulate pharmaceutical excipient. Also disclosed are granules, a suppository for rectal administration, and a capsule filled with the granules. Methods for preparing the tablet, the suppository and the granules are also disclosed as well as uses of the granules and a method for coating them.

### FIELD OF THE INVENTION

The present invention relates to a pharmaceutical tablet comprising a lipid phase, lipid granules for making the tablet and of independent use, such as for filling of capsules, and processes for preparing the tablet and the granules and for coating the granules. The present invention also relates to a pharmaceutical suppository and a method for its preparation.

### BACKGROUND OF THE INVENTION

There is a need for better control of gastro-intestinal absorption of drugs, in particular drugs administered in tablets or suppositories comprising a lipid phase.

Tablets comprising a lipid phase are known in the art. WO 03/061627 A1 discloses a process for the preparation of a self-dispersing or self-emulsifying tablet comprising the steps of mixing a granulation medium containing an active lipophilic substance with non-swellable fillers and optionally binders, granulating the mixture, drying the granules obtained, sieving the granules into a size below 1 mm, mixing the granules with tabletting aids, and compressing the mixture into tablets. The granulation medium of WO 03/061627 A1 comprises an oil, a surfactant, in particular fatty acid esters of glycerol and of polyethylene glycol. If the oil (fat) content of the tablet formulation exceeds 20% a binder such as polyvinyl pyrrolidone has to be used.

### OBJECTS OF THE INVENTION

It is an object of the present invention to provide a pharmaceutical tablet or a pharmaceutical suppository comprising a lipid phase and a pharmacologically active agent, the gastrointestinal absorption of which can be controlled over a wide range, so as to obtain a desired concentration of the active agent and/or metabolites thereof in plasma.

It is another object of the invention to provide lipid granules for making the tablet or the suppository.

It is an additional object of the invention to provide a pharmaceutical tablet or suppository comprising a lipid phase, into which a pharmacologically active agent that is not soluble in the lipid phase can be integrated, and to provide corresponding granules that can be compressed to form the tablet.

Further objects of the invention include processes for making the tablets, suppositories, and granules of the invention.

Still further objects of the invention will be realized by studying the following summary of the invention, the description of preferred embodiments thereof, and the appended claims.

### SUMMARY OF THE INVENTION

The gastrointestinal absorption of a drug in a lipid pharmaceutical composition is controlled, i.a., by the nature and amount of its lipid excipients. The present invention is based on the insight that, in addition to such control, the gastrointestinal absorption of a drug, in particular a lipophilic drug, can be controlled varying the nature and/or amount of particulate pharmaceutical excipients comprised by the composition and which are insoluble in the composition. Such control is of particular interest for adapting the bioavailability profile of the composition to that of traditional non-lipid compositions and for enhancing gastrointestinal absorption in general.

According to the present invention is disclosed a tablet for oral administration comprising a lipid phase, preferably a continuous lipid phase, comprised to 80% by weight or more by a mixture of (a) triglyceride (b) mono- or/and diglyceride, and (c) cell membrane lipid; (d) one or more pharmacologically active agents dissolved or dispersed, preferably dispersed, in the lipid phase; (e) water and/or ethanol; (f) an absorption controlling amount of particulate pharmaceutical excipient. The term “dissolved or dispersed in the lipid phase” includes active agents partially dissolved and partially dispersed in the lipid phase.

It is preferred for the triglyceride to have a solid fat content at body temperature. It is preferred for the lipid phase to essentially consist of triglyceride, preferably triglyceride having a solid fat content at body temperature, monoglyceride, and cell membrane lipid. It is understood that the composition of the lipid phase as stated in the foregoing is exclusive of the pharmacologically active agent(s) dissolved or dispersed therein.

It is preferred for the lipid phase to comprise from 40% by weight to 95% by weight of triglyceride, from 1% by weight to 35% by weight of mono- and/or diglyceride, from 0.5% by weight to 40% by weight of membrane lipid, with the proviso that the weight percentages of these components add up to 90% or more, preferably to about 100% of the lipid phase.

The triglyceride (triacylglycerol) of the invention can be any triglyceride material. The solid fat content, if any, can be determined by NMR serial measurements as described in IUPAC method no. 2150, 7^{th} edition. The triglyceride is preferably selected from edible oils of animal and/or vegetable origin and/or fractions thereof, such as the soybean oil, palm oil, palm kernel oil, corn oil, sunflower oil, cocoa butter, lard, tallow, and palm olein. Further examples of triglyceride are illipe butter, shea butter, kokum butter, sal butter and other natural oils or fractions thereof. Other examples of triglyceride oils include hydrogenated or partially hydrogenated triglyceride oil selected from partially or fully hydrogenated soybean oil, rapeseed oil, cotton oil, sunflower oil, and fractions thereof. The triglyceride oil may be synthetic or semi-synthetic, such as medium-chain triglyceride oil (MCT). It is understood that the triglyceride of the invention is an edible oil or a mixture of two or more edible oils, in particular of the aforementioned oils. The triglyceride of the invention preferably contains 95% by weight or more of triacylglycerol, preferably 98% or more, most preferred 99% or more.

The mono- and diglyceride of the invention is preferably selected from fatty acid ester of glycerol and fatty acid ester of polyethylene glycol and their mixtures. Particularly preferred is mono- and/or diglyceride selected from glycerol esters of C₈-C₁₈ fatty acids; also preferred are macrogol esters of C₈-C₁₈ fatty acids. Even more preferred is mono- and glyceride and mixtures of monoglyceride and/or diglyceride selected from C₁₀ and C₁₂ fatty acid esters of glycerol as well as mixtures of mono- and/or diglyceride comprised of C₁₀ and C₁₂ fatty acid esters of glycerol by more than 50% by weight, preferably more than 80% by weight.

The cell membrane lipid of the invention is preferably selected from glycolipid, phospholipid and sphingolipid. Most preferred is glycolipid, in particular galactolipid, most preferred digalactosyl-diacylglycerol.

The pharmacologically active agent of the invention can be any agent that is sufficiently soluble in the continuous lipid phase of the invention and/or dispersible in it to provide for per-oral administration of a pharmacologically effective amount thereof in a tablet. In this application “particulate pharmaceutical excipient” is a traditional particulate pharmaceutical tablet excipient selected from filler, binder, glidant, anti-adherent, lubricant, disintegrant, anti-oxidant, colorant, flavouring and their mixtures. It is understood that the particulate pharmaceutical excipient is essentially insoluble in the continuous lipid phase. It is preferred for the particulate pharmaceutical excipient to comprise or to consist of material of a high surface to weight ratio, such as amorphous silicon dioxide; the surface to weight ratio being preferably more than 0.5 m²/g, more preferred 1 m²/g, most preferred more than 2 m²/g. Preferably the particulate pharmaceutical excipient is in powderous form and comprises one or more of amorphous silicon dioxide, which is most preferred, titanium dioxide, aluminium oxide, basic aluminium oxide, calcium sulphate, calcium carbonate, microcrystalline cellulose, dibasic calcium phosphate dihydrate, tribasic calcium phosphate, microcrystalline cellulose, powdered cellulose, cyclodextrins, bentonite, kaolin, lactose, magnesium aluminium silicate, magnesium carbonate, magnesium oxide, magnesium trisilicate, and talc.

“Powderous form” signifies a particle size of preferably less than 400 μm, more preferred less than 200 μm, even more preferred less than 100 μm, of 80% or more by weight of the excipient particles, preferably of 90% or more by weight of the excipient particles.

It is preferred for the water and/or ethanol of the tablet of the invention to be comprised by, preferably dissolved in, the lipid phase.

The tablet of the invention can be formed by, for instance, pouring the molten lipid phase, preferably a molten continuous lipid phase, into a mould, and let it solidify in the mould.

Alternatively and particularly preferred, the molten lipid phase, in particular a molten continuous lipid phase, is granulated, and the lipid granules so obtained are compressed into tablets with or without the use of non-lipid powderous pharmaceutical excipient, in particular pharmaceutical tabletting excipient such as magnesium stearate and colloidal silica. The tablet or suppository of the invention obtained by compressing a mixture of lipid granules and tabletting excipient will consist of deformed lipid granules adhering to each other and non-lipid tabletting excipient inhomogeneously distributed within the tablet, in particular near the boundaries between deformed lipid granules; optionally it may comprise a coating such as a sugar coating.

According to the present invention is also disclosed a method of preparing a tablet for oral administration comprising a pharmacologically effective amount of a drug dissolved and/or dispersed, preferably dispersed, in a lipid phase, comprising:

mixing from 40 parts by weight to 95 parts by weight of triglyceride, from 1 part by weight to 35 parts by weight of mono- and/or diglyceride, from 0.5 parts by weight to 40 parts by weight of membrane lipid at a temperature sufficient to melt any solid triglyceride to obtain a continuous lipid phase, with the proviso that the parts by weight shall add up to 100;

dispersing water and/or ethanol in an amount of from 1% by weight to 10% by weight of the lipid phase;

dispersing a selected amount of a pharmacologically active agent in the lipid phase;

providing, in a separate container, powderous pharmaceutical excipient;

adding over a selected period of time the lipid phase containing the active agent to the powderous pharmaceutical excipient under vigorous agitation;

allowing the mass to cool to ambient temperature under continued agitation to obtain a granular product;

sieving the granular product to obtain a desired granule fraction;

optionally mixing the granule fraction with pharmaceutical tabletting excipient;

compressing aliquots of the granule fraction or the mixture of granule fraction and pharmaceutical tabletting excipient to tablets.

It is understood that the steps of dispersing water and/or ethanol, of dispersing the pharmacologically active agent, and of adding the lipid phase containing the active agent to the powderous pharmaceutical excipient are carried out at a temperature at which the solid triglyceride is in a molten state; if necessary the water and/or ethanol and/or the active agent and/or the powderous pharmaceutical excipient are brought to such temperature prior to dispergation or addition, respectively.

Alternatively, a lipophilic pharmacologically active agent may be dissolved in the lipid phase prior to forming the dispersion.

It is preferred for the mixing of the lipid components to be carried out at a temperature of 50° C. or more, preferably at a temperature of from 60° C. to 75° C.

According to a preferred aspect of the invention the method of preparing an oral tablet can be stopped at the granule fraction stage, thus providing a process for production of lipid granules. The lipid granules of the invention, which share the components of the tablet of the invention except for, if present, tabletting excipient(s), can be separately transformed to tablets for oral administration, used for filling gelatine or other kind of capsules, be coated, be formed into suppositories, and the like. It is understood that the features of the constituents of the tablet of the invention such as, for instance, the nature of the triglyceride material, the nature of the mono- and/or diglyceride material, the nature of the cell membrane lipid, the nature of the powderous pharmaceutical excipient, also apply to the corresponding constituents used in the process of the invention for producing a tablet or granules.

Thus, according to a preferred aspect of the invention are also disclosed granules comprising a lipid phase, preferably a continuous lipid phase, comprised to 80% by weight or more by a mixture of (a) triglyceride (b) mono- or/and diglyceride, and (c) cell membrane lipid; (d) one or more pharmacologically active agents dissolved or dispersed, preferably dispersed, in the lipid phase; (e) water and/or ethanol; and (f) an absorption controlling amount of particulate pharmaceutical excipient. The lipid phase of the granule shares the advantageous features of the tablet of the invention, which do not need to be repeated here.

The granules of the invention can advantageously be coated, for instance by pan coating or spray coating. The coating thus produced may comprise an enteric layer.

Furthermore disclosed is the use of the granules according to the invention for forming a suppository.

In this application ambient temperature is a temperature of from about 18° C. to about 24° C., body temperature is a temperature of about 37° C.

The invention will now be explained in more detail by reference to a number of preferred embodiments. The examples are only provided for the purpose of illustration, and should not be understood as limiting the invention in any respect.

### DESCRIPTION OF PREFERRED EMBODIMENTS

### Example 1

Preparation of lipid mixtures. Ten lipid mixtures (10 g each) were prepared from the ingredients listed below by mixing and stirring the components in a glass beaker at a temperature of about 50° C.

Ingredients: CPL galactolipid, LTP Lipid Technologies Provider AB, Karlshamn, Sweden; galactolecithin (fractionated oat oil), LTP Lipid Technologies Provider AB, Karlshamn, Sweden; Akoline MCM (medium chain monoglyceride), Karlshamns AB, Karlshamn, Sweden; MCM (fractionated medium chain monoglyceride), LTP Lipid Technologies Provider AB, Karlshamn, Sweden; triglyceride oils; Karlshamns AB, Karlshamn, Sweden.

TABLE 1
Exemplary lipid mixtures of the invention
(all components given in % by weight)

| | Ako- | | CPL | Galacto- | |
|---|---|---|---|---|---|
| Lipid | line | | Galacto- | leci- | Triglyceride |
| mixture # | MCM*) | MCM | lipid**) | thin***) | oil |
| G1 | 50 | | | 50 | |
| G2 | | 50 | | 50 | |
| G3 | 66.7 | | | 33.3 | |
| G4 | | 66.7 | | 33.3 | |
| G5 | 15 | | 20 | | 65 (palmkernel |
| | | | | | stearin) |
| G6 | | 80 | 20 | | |
| G7 | | 22.9 | | 77.1 | |
| G8 | | | | 100 | |
| G9 | | 10 | 50 | | 40 (palm oil) |
| G10 | | 10 | 50 | | 40 (MCT oil) |

*)Contains diglyceride.
**)Described in WO 95/20943; contains about 50% by weight of triglyceride.
***)Described in WO 97/11141.

Preparation of lipid mixture G5. A stock lipid mixture (10 kg) was prepared from 20% by weight of galactolipid (CPL galactolipid, LTP Lipid Technologies Provider AB, Karlshamn, Sweden), 15% by weight of medium chain monoglyceride (Alkoline MCM, Karlshamns AB, Karlshamn, Sweden), and triglyceride (Palmkernel stearin, Karlshamns AB) by mixing and stirring the components in a glass vessel at a temperature of about 50° C. The other exemplary lipid mixtures in Table 1 were also prepared by this method.

### Example 2

Preparation of granules containing a single pharmaceutical excipient. Lipid mixture G5 (10 g) at 40° C. was added to 10 g of isomalt in a glass beaker under agitation. After completion of addition the contents were allowed to cool to room temperature under continued agitation. The solidified granules were passed through a 710 μm sieve.

This process was successfully repeated for each of the following excipients: isomalt; mannitol; lactose; pregelled starch; native starch; talcum; magnesium stearate; amorphous silica; polyvinylpyrrolidone; croscarmellose sodium; MCC PH-102, except for the amount of amorphous silica being 5 g.

Preparation of granules containing two pharmaceutical excipients from lipid mixtures G1 to G10. Each lipid mixture G1 to G10 (10 g) at 40° C. was added separately under agitation to 10 g of a premixed powder consisting of 75% by weight of micro-crystalline cellulose (MCC PH102, FMC Corporation, Cork, Ireland) and of 25% by weight of colloidal silicon dioxide (Aerosil® 200; Degussa, Frankfurt, Germany). After completion of addition the contents were allowed to cool to room temperature under continued agitation. The solidified granules were passed through a 710 μm sieve.

Preparation of granules used for making tablets GT1 and GT2. To lipid mixture G5 (950 g) at 70° C. in a glass beaker was added approx. 5% by weight of water under mixing for 4 min followed by adding 0.7 g of vitamin B12 (cyanocobolamine) while stirring until dissolution. In another beaker 164 g of micro-crystalline cellulose (MCC PH102, FMC Corporation, Cork, Ireland) and 55 g of colloidal silicon dioxide (Aerosil® 200; Degussa, Frankfurt, Germany) [GT1] or 164 g of micro-crystalline cellulose [GT2] were pre-mixed for 3 min to break up agglomerates. The lipid mixture was cooled to 40° C. and slowly added under agitation to the premixed MCC/SiO₂ [GT1] or MCC [GT2] powder. After complete addition the contents were allowed to cool to room temperature under continued agitation. The solidified granules were passed through a 710 μm sieve.

### Example 3

Preparation of tablets. From the sieved granules of Example 2 tablets termed GT1 and GT2 were prepared. For the preparation of tablets GT1 granules of Example 3 were mixed with Isomalt DC-100 (stoichiometric mixture of 6-O-α-D-glucopyranosyl-D-sorbitol and 1-O-α-glucopyranosyl-D-mannitol dehydrate; Palatinit GmbH, Mannheim, Germany), HPMC (hypromellose; Shin-Etsu Chemical Comp., Ltd, Tokyo, Japan), magnesium stearate (Peter Greven Nederland C.V., Venloo, Netherlands) and Aerosil® 200 (Degussa AB, Frankfurt, Germany) in the proportions given in Table 2. For the preparation of tablets GT2 the granules of Example 3 were used without additives.

The particulate mixtures GT1 and GT2 were filled in a hopper of a single punch tablet machine to produce convex tablets with a diameter of 13 mm and a total weight of 700 mg (GT1) and 507.5 mg (GT2).

TABLE 2
Composition of tablet preparations GT1 and GT2

| | |
|---|---|
| GT1 | |
| Amount per tablet | GT2 |

| | | | |
|---|---|---|---|
| Component | (mg) | (%) | Amount per tablet |

| | | | | |
|---|---|---|---|---|
| Cyanocobolamine | 0.5 | 0.07 | 0.5 | 0.1 |
| Lipid mixture G5 | 119.7 | 17.1 | 198.5 | 39.1 |
| Purified water | 6.3 | 0.9 | 9.9 | 2.0 |
| | 126.5 | | 208.9 | |
| MCC PH102 | 115.1 | 16.4 | 298.6 | 58.8 |
| Aerosil 200 | 38.4 | 5.5 | — | — |
| | 280.0 | | 298.6 | |
| Isomalt DC-100 | 379.6 | 54.2 | — | — |
| HPMC | 35.0 | 5.0 | — | — |
| Mg stearate | 3.5 | 0.5 | — | — |
| Aerosil 200 | 1.9 | 0.3 | — | — |
| | 700.0 | 99.97 | 507.5 | 99.9 |

In Table 2 granule components are shown in fat style while tabletting excipients are shown in fat italic style.

### Example 4

Gastrointestinal absorption in humans. Five healthy male volunteers who had fasted for 10 hours were given a commercial vitamin B12 preparation (Behepan®; Pharmacia; 2 mg active substance) as a reference administration. The serum concentration of vitamin B12 obtained by the reference composition was compared with corresponding administrations in which the same volunteers had been given 2.0 mg vitamin B12 in form of GT1 or GT2 tablets. Thus each subject became his own control.

Blood samples were collected from the volunteers prior to administration and at 1, 2, 3, 4, and 8 hours after administration. Serum concentrations of vitamin B12 were determined at the Laboratory of Clinical Chemistry, Karolinska University Hospital, Huddinge (Table 2).

The pre-administration values are regarded as baseline. The baseline value for each subject was subtracted from each of the sampling points. The area under the curve (AUC) was calculated by the linear trapezoidal method to the last blood concentration. For each subject the reference tablet AUC was compared with the AUC of the tablets of the invention (GT1 and GT2).

TABLE 2
Plasma concentration of vitamin B12 obtained by administration
of GT1, GT2, and reference tablets

| Time | Subject A | | | Subject B | | | Subject C | | Subject D | | Subject E | |
|---|---|---|---|---|---|---|---|---|---|---|---|---|
| (h) | Ref. | GT1 | GT2 | Ref. | GT1 | GT2 | Ref. | GT1 | Ref. | GT2 | Ref. | GT2 |
| 0 | 271 | 381 | 430 | 233 | 262 | 276 | 188 | 154 | 183 | 226 | 352 | 387 |
| 1 | 286 | 410 | 503 | 262 | 334 | 464 | 349 | 234 | 239 | 556 | 425 | 704 |
| 2 | 322 | 373 | 519 | 303 | 383 | 383 | 404 | 281 | 295 | 639 | 451 | 701 |
| 3 | 313 | 347 | 576 | 317 | 380 | 364 | 449 | 320 | 293 | 567 | 462 | 816 |
| 4 | 324 | 357 | 565 | 317 | 257 | 386 | 377 | 312 | 311 | 545 | 481 | 85 |
| 6 | 329 | 351 | 612 | 311 | 277 | 390 | 410 | 299 | 285 | 506 | 461 | 940 |
| 8 | 337 | 384 | 635 | 254 | 408 | 408 | 428 | 218 | 288 | 487 | 460 | 892 |
| AUC | | 9 | 292 | | 71 | 134 | | 75 | | 306 | | 426 |
| % of | | | | | | | | | | | | |
| Ref. | | | | | | | | | | | | |

1. A tablet for oral administration comprising a lipid phase comprised to 80% by weight or more by a mixture of triglyceride, mono- or diglyceride or both, and cell membrane lipid; one or more pharmacologically active agents dissolved or dispersed or both in the lipid phase; water or ethanol or both; and an absorption controlling amount of particulate pharmaceutical excipient.

2. The tablet of claim 1, comprising from 1% to 10% by weight of the water or ethanol or both.

3. The tablet of claim 1, wherein the triglyceride has a solid fat content at body temperature.

4. The tablet of claim 1, wherein the lipid phase comprises from 40% by weight to 95% by weight of triglyceride, from 1% by weight to 35% by weight of mono- or diglyceride or both, from 0.5% by weight to 40% by weight of membrane lipid, with the proviso that the weight percentages of these components add up to 90% or more.

5. (canceled)

6. The tablet of claim 3, wherein the lipid phase comprises from 40% by weight to 95% by weight of triglyceride, from 1% by weight to 35% by weight of mono- or diglyceride or both, from 0.5% by weight to 40% by weight of membrane lipid, with the proviso that the weight percentages of these components add up to 100%.

7. The tablet of claim 1, wherein the triglyceride comprises an edible oil of animal or vegetable origin, hydrogenated or partially hydrogenated oil thereof, fractions thereof, and mixtures thereof; and wherein the cell membrane lipid is a cell membrane lipid from glycolipid, phospholipid and sphingolipid.

8. The tablet of claim 7, wherein triglyceride is selected from the group consisting of soybean oil, palm oil, palm kernel oil, sunflower oil, cocoa butter, lard, tallow, palm olein, illipe butter, shea butter, kokum butter, sal butter, hydrogenated or partially hydrogenated soybean oil, hydrogenated or partially hydrogenated rapeseed oil, hydrogenated or partially hydrogenated corn oil, hydrogenated or partially hydrogenated cotton oil, hydrogenated or partially hydrogenated sunflower oil, fractions thereof, and mixtures thereof; and wherein the cell membrane is glycolipid.

9-10. (canceled)

11. The tablet of claim 1, wherein the mono- or diglyceride is selected from fatty acid ester of glycerol and fatty acid ester of polyethylene glycol.

12. (canceled)

13. The tablet of claim 10, wherein the mono- or diglyceride is selected from C₁₀ and C₁₂ fatty acid esters of glycerol and mixtures of mono- and diglyceride comprised of C₁₀ and C₁₂ fatty acid esters of glycerol by more than 50% by weight.

14-15. (canceled)

16. The tablet of claim 7, wherein the glycolipid comprises galactolipid.

17. The tablet of claim 16, wherein the galactolipid comprises digalactosyl-diacylglycerol.

18-19. (canceled)

20. The tablet of claim 1, consisting of deformed lipid phase granules and non-lipid tabletting excipient inhomogeneously distributed within the tablet and, optionally, a coating.

21-22. (canceled)

23. The method of claim 32, wherein the combining of the lipid components is carried out at a temperature of 50° C. or more.

24. The method of claim 23, wherein the mixing of the lipid components is carried out at a temperature of from 60° C. to 75° C.

25-26. (canceled)

27. The method of claim 32 further comprising filling capsules capable of disintegrating in gastrointestinal fluid with said granules.

28. A capsule capable of disintegrating in gastrointestinal fluid filled with granules prepared by the method of claim 32.

29. The method of claim 32 further comprising pan coating or spray coating said granules.

30. The method of claim 29, wherein the coating comprises an enteric layer.

31. The method of claim 32 further comprising forming a suppository with said granules.

32. A method of preparing a composition for oral administration comprising a pharmacologically effective amount of a drug dissolved or dispersed or both in a lipid phase, the method comprising:
combining (a) a continuous lipid liquid phase comprising from 40 parts by weight to 95 parts by weight of triglyceride, from 1 part by weight to 35 parts by weight of mono- or diglyceride or both, from 0.5 parts by weight to 40 parts by weight of membrane lipid with the proviso that the parts by weight add up to 100, (b) water or ethanol or both in an amount of from 1% by weight to 10% by weight of the lipid phase, and (c) a pharmacologically active agent, wherein the active agent is combined with the liquid phase after the water or ethanol is combined therewith or the active agent is lipophilic and combined with the liquid phase before the water or ethanol is combined therewith;
providing a powderous pharmaceutical excipient;
combining the lipid phase containing the pharmacologically active agent with the powderous pharmaceutical excipient under agitation;
allowing the mass to cool to ambient temperature under agitation to obtain a granular product; and
optionally, sieving the granular product to obtain a desired granule fraction.

33. The method of claim 32, further comprising compressing aliquots of the granules or a mixture thereof with pharmaceutical tabletting excipient to tablets.
